(19) United States
(12) Patent Application Publication
(10) Pub. No.: US 2010/0280508 A1
(43) Pub. Date: Nov. 4, 2010

(54) Method and Apparatus for RF Anastomosis

(76) Inventor: Joseph Charles Eder, Los Altos Hills, CA (US)

Correspondence Address: SHAY GLENN LLP, 2755 CAMPUS DRIVE, SUITE 210, SAN MATEO, CA, 94403, US

(21) Appl. No.: 12/548,765

(22) Filed: Aug. 27, 2009

Related U.S. Application Data
(60) Provisional application No. 61/174,866, filed on May 1, 2009.

(51) Int. Cl. A61B 18/18

(52) U.S. Cl. 606/33

(57) ABSTRACT

RF electrocautery is used to create an anastomosis between vessels or organs, such as the bowel. In an embodiment, tissues are fused together to create a seal between two or more tissue surfaces, for example in a side-to-side bowel anastomosis.

### CROSS REFERENCE TO RELATED APPLICATIONS

This application claims priority to U.S. provisional patent application Ser. No. 61/174,866, filed 1 May 2009, which application is incorporated herein in its entirety by this reference thereto.

### BACKGROUND OF THE INVENTION

1. Technical Field

The invention relates to electrocautery. More particularly, the invention relates to a method and apparatus for RF anastomosis.

2. Description of the Prior Art

Anastomosis is the connection of two structures. It refers to connections between blood vessels or between other tubular structures, such as loops of intestine. In circulatory anastomosis, many arteries naturally anastomose with each other, for example the inferior epigastric artery and superior epigastric artery. The circulatory anastomosis is further divided into arterial and venous anastomosis. Arterial anastomosis includes actual arterial anastomosis, e.g. palmar arch and plantar arch, and potential arterial anastomosis, e.g. coronary arteries and cortical branch of cerebral arteries. An example of surgical anastomosis occurs when a segment of intestine is resected and the two remaining ends are sewn or stapled together (anastomosed), for example Roux-en-Y anastomosis. This procedure is referred to as intestinal anastomosis. Such procedures have heretofore relied upon conventional surgical practices which are highly invasive, time-consuming, and complex. It would be advantageous to provide a simpler procedure for performing anastomosis.

### SUMMARY OF THE INVENTION

An embodiment of the invention provides a method and apparatus for RF electrocautery that creates an anastomosis between vessels or organs, such as the bowel. In an embodiment, tissues are fused together to create a seal between two or more tissue surfaces, for example in a side-to-side bowel anastomosis.

### BRIEF DESCRIPTION OF THE DRAWINGS

FIG. 1 is a perspective view of a diseased bowel as is known in the art;

FIG. 2 is a perspective view of a first step in a side-to-side bowel anastomosis in which an RF device is used seal and dissect both ends of a diseased bowel according to the invention;

FIG. 3 is a perspective view of a second step in a side-to-side bowel anastomosis in which a diseased bowel is removed according to the invention;

FIG. 4 is a perspective view of a third step in a side-to-side bowel anastomosis in which openings are created in both sealed ends of a bowel to accommodate the jaws of an RF device according to the invention;

FIG. 5 is a perspective view of a fourth step in a side-to-side bowel anastomosis in which an RF device is inserted into openings in both ends of a bowel to create a sealed region therebetween and to thereafter dissect a portion of the sealed region according to the invention;

FIG. 6 is a top view of a bowel after the bowel is sealed and cut, as shown in FIG. 5, according to the invention;

FIG. 7 is a perspective view of a fifth step in a side-to-side bowel anastomosis in which an RF device is used seal both ends of a diseased bowel according to the invention; and

FIG. 8 is a perspective view of an anastomosed bowel according to the invention.

### DETAILED DESCRIPTION OF THE INVENTION

An embodiment of the invention provides a method and apparatus for RF electrocautery that creates an anastomosis between vessels or organs, such as the bowel. In an embodiment, tissues are fused together to create a seal between two or more tissue surfaces, for example in a side-to-side bowel anastomosis.

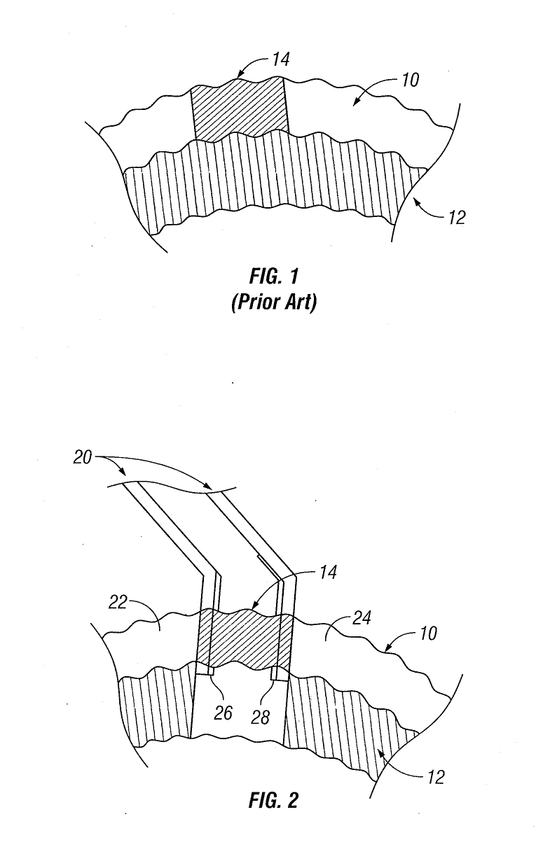

FIG. 1 is a perspective view of a bowel 10 and mesentery (connecting tissue) 12, showing a diseased segment 14 of the bowel.

FIG. 2 is a perspective view of a first step in a side-to-side bowel anastomosis in which an RF device 20 is used to seal and dissect two portions 22, 24 of a bowel having a diseased segment 14 therebetween. The RF device is used separately in two different operations as part of this step in the procedure. The mesentery that supplies the diseased segment of the bowel is first sealed and removed using the RF device. The RF device is then used to seal and dissect both ends of the bowel to isolate and remove the diseased segment. The bowel ends are sealed by the application of RF energy to the bowel on either side of the diseased bowel segment through a pair of electrode-bearing jaws. A blade within the RF device, or other cutting means, is used to cut the diseased segment away from the healthy, sealed portion of the bowel. These operations may be performed simultaneously or in sequence. The RF device used for this procedure may be of a type, for example, disclosed in any of pending U.S. patent application Ser. No. 11/382,635 (filed 10 May 2006), Ser. No. 11/193,881 (filed 2 May 2007), Ser. No. 11/382,652 (filed 10 May 2006), Ser. No. 11/671,891 (filed 6 Feb. 2007), Ser. No. 11/671,911 (filed 6 Feb. 2007), and Ser. No. 12/027,231 (filed 6 Feb. 2008), each of which is incorporated herein in its entirety by this reference thereto; or it may any other of such devices as are suited for this procedure.

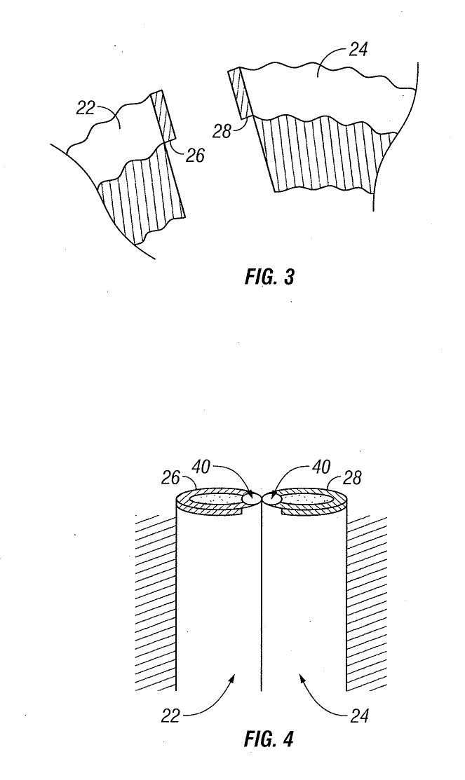

FIG. 3 is a perspective view of a second step in a side-to-side bowel anastomosis in which a diseased bowel is removed. In FIG. 3, the ends 22, 24 of the healthy portions of the bowel are shown. Each end of the bowel has been sealed 26, 28 by the RF device.

FIG. 4 is a perspective view of a third step in a side-to-side bowel anastomosis in which openings 40 are created in both sealed ends 26, 28 of the bowel to accommodate the jaws of the RF device. The opening are created, for example, by cutting corners out of the fused end regions of the bowel segments. These openings may be made by conventional means, such as by scalpels or other such known devices.

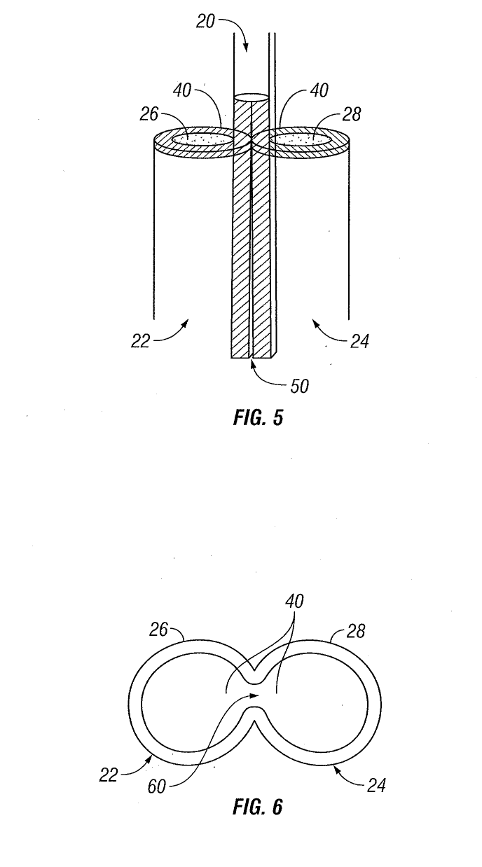

FIG. 5 is a perspective view of a fourth step in a side-to-side bowel anastomosis in which the RF device 20 is inserted into the openings 40 in both ends of the bowel to create a sealed region 50 therebetween and to thereafter dissect a portion of the sealed region along the bowel axis. Thus, the RF device is inserted into the openings in the end of the bowel segments and clamped shut with opposing electrodes thereof facing the walls of the bowel segments that are in contact with each other. RF energy is applied to the electrodes and the bowel ends are joined together along an axis thereof. A cutting blade in the RF device is then used to create an opening within the joined region of the bowel segments.

FIG. 6 is a top view of a bowel after the bowel is sealed and cut, as shown in FIG. 5. In FIG. 6, it can be seen that the two bowel segments 22, 24 have been joined axially and that the joined region is bisected to form a passage 60 therebetween.

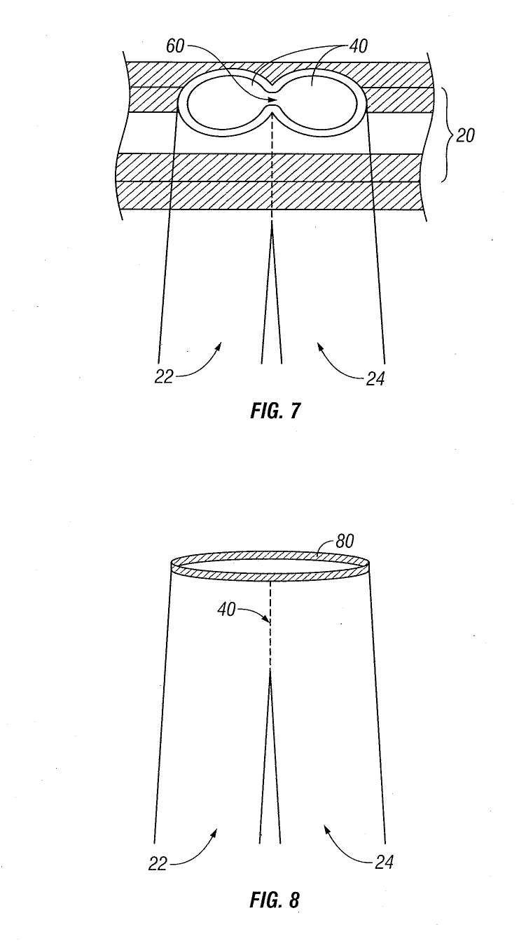

FIG. 7 is a perspective view of a fifth step in a side-to-side bowel anastomosis in which the RF device 20 is used to seal both ends of the bowel. As a result, the two healthy portions of the bowel have been joined in a side-to-side fashion, an opening therebetween has been created along the join, and the ends thereof have been sealed, thus providing a fully functioning bowel after excision of the diseased portion thereof.

FIG. 8 is a perspective view of an anastomosed bowel.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A method for creating an anastomosis between vessels or organs, comprising the steps of:
fusing tissues together with RF electrocautery to create a seal between two or more tissue surfaces; and
making an opening between said tissue surfaces at a location at which said tissues are fused together to allow fluid communication between structures defined by said two or more tissue surfaces.

2. The method of claim 1, further comprising the step of:
performing said fusing and opening steps to create a side-to-side bowel anastomosis.

3. A method for creating a side-to-side bowel anastomosis, comprising the steps of:
using an RF device to seal and dissect two portions of a bowel having a diseased segment therebetween;
removing the diseased bowel segment;
creating openings in the sealed end of each of the two portions of the bowel to accommodate the jaws of said RF device;
inserting the jaws of said RF device into the openings in both ends of the bowel, clamping the jaws of the RF device together with opposing electrodes thereof facing walls of the two portions of the bowel, and applying RF energy to create a sealed region therebetween; and
dissecting a portion of the sealed region along a bowel axis to create an opening between the two portions of the bowel to allow fluid communication therebetween; and
using the RF device to seal both ends of the bowel;
wherein two healthy portions of the bowel are communicatively joined in a side-to-side fashion.

4. The method of claim 3, further comprising the step of:
using a cutting blade in the RF device to create said opening between the two portions of the bowel.

5. The method of claim 3, further comprising the step of:
creating said openings in the sealed end of each of the two portions of the bowel by cutting corners out of the sealed end regions thereof.

6. The method of claim 3, further comprising the step of:
using said RF device to seal and remove a mesentery that supplies said diseased segment of the bowel.

7. The method of claim 3, said step of using the RF device to seal and dissect both ends of the bowel to isolate and remove the diseased segment further comprising the steps of:
sealing the ends of each of the two portions of the bowel by the application of RF energy to the bowel on either side of the diseased bowel segment through a pair of electrode-bearing jaws; and
using a blade within the RF device to cut the diseased segment away from the two sealed portions of the bowel.

8. An apparatus for creating an anastomosis between vessels or organs, comprising:
an RF device having a pair of jaws comprising opposed electrode surfaces that are configured for fusing tissues together with RF electrocautery to create a seal between two or more tissue surfaces; and
means within said RF device for making an opening between said tissue surfaces at a location at which said tissues are fused together to allow fluid communication between structures defined by said two or more tissue surfaces.

9. The apparatus of claim 8, wherein said RF device is adapted for use to create a side-to-side bowel anastomosis.

10. An apparatus for creating a side-to-side bowel anastomosis, comprising:
an RF device having a pair of jaws comprising opposed electrode surfaces that are configured to seal and dissect two portions of a bowel having a diseased segment therebetween;
said RF device jaws being configured for insertion into the openings in both ends of the bowel, said RF device jaws being operable to clamp the jaws of the RF device together with opposing electrodes thereof facing walls of the two portions of the bowel, and said electrodes being operable to apply RF energy to create a sealed region therebetween; and
said RF device comprising a cutting device for dissecting a portion of the sealed region along a bowel axis to create an opening between the two portions of the bowel to allow fluid communication therebetween; and
said RF device being configured to seal both ends of the bowel;
wherein two healthy portions of the bowel are communicatively joined in a side-to-side fashion.

11. The apparatus of claim 10, further comprising:
a cutting blade in the RF device to create said opening between the two portions of the bowel.

12. The apparatus of claim 10, the RF device configured to seal the ends of each of the two portions of the bowel by the application of RF energy to the bowel on either side of the diseased bowel segment through a pair of electrode-bearing jaws; and
a blade within the RF device for cutting the diseased segment away from the two sealed portions of the bowel.
